(19) United States
(12) Patent Application Publication
(10) Pub. No.: US 2004/0115240 A1
(43) Pub. Date: Jun. 17, 2004

(54) Composite for attaching, growing and/or repairing of living tissues and use of said composite

(76) Inventors: Timo Narhi, Helsinki (FI); Antti Yli-Urpo, Littoinen (FI); Pekka Vallittu, Kuusisto (FI); Marju Vakiparta, Turku (FI); Teemu Tirri, Turku (FI); Mervi Puska, Turku (FI); Lippo Lassila, Turku (FI); Tapani Lastumaki, Helsinki (FI); Allan Ho, Turku (FI)

Correspondence Address: James C Lydon, Suite 100, 100 Daingerfield Road, Alexandria, VA, 22314, US

(21) Appl. No.: 10/472,112

(22) PCT Filed: Mar. 12, 2002
(86) PCT No.: PCT/FI02/00192

(30) Foreign Application Priority Data
Mar. 16, 2001 (FI) 2001-0540

(51) Int. Cl. A61F 2/00

(52) U.S. Cl. 424/426

(57) ABSTRACT

The invention relates to a composite for attaching, growing and/or repairing of living tissue in mammals. The composite comprises a non-expandable matrix polymer and a water-expandable porosity agent. The invention also relates to the use of said composite.

### FIELD OF INVENTION

The present invention relates to a composite for attaching, growing and/or repairing of living tissues in mammals. The invention further relates to the use of said composite.

### BACKGROUND OF THE INVENTION

Different resorbable materials have been used for the treatment of tissue defects in otolaryngological, dental, orthopedic and plastic surgery. Autogenous bone and soft tissue transplants are mostly used. However, the donor site morbidity and the limited amount of tissue available restrict their use. An additional surgical procedure is also usually needed for harvesting the tissue transplant. Autologous tissue transplants, e.g. bank bone, have widely been used, although unwanted immunological reactions restrict their use. The use of synthetic organic and inorganic materials is therefore rapidly increasing. Their advantages are that large amounts of these materials can be rapidly produced, their properties can be tailored according to the clinical requirements and there is no or at least considerably less unwanted immunological reactions compared to autologous tissue transplants.

Among others, thermoplastic bioabsorbable polymers, copolymers and their composites are potential materials in the treatment of various soft and hard tissue defects. An optimal material induces and conducts tissue regeneration while it simultaneously degrades during the healing of the target tissue. Speed of degradation ought to be determined by the regenerative capacity of the target tissue in question.

Several biodegradable polymeric materials have been developed for medical applications. Most materials are polyester derivates, of which polylactide and caprolactone are best documented. These polymers are currently considered as biocompatible, non-toxic materials. Certain polyester copolymers (ε-caprolactone-D, L-lactide) can remain moldable in low temperatures, which make it possible to inject them into tissue defects as disclosed in WO 99/02211 (Aho et al.).

Also several composites comprising polymer(s) are designed for medical applications in order to improve the contact between the living tissue and the composite. The connection between the composite and the living tissue is normally only mechanical, because the structure of the composite is usually too dense after implantation and does not allow any place for new tissue ingrowth inside the composite material. Therefore, the contact area between the composite and the living tissue is only limited to the contact surface between them. A porous material would solve this problem by providing a larger contact area between the tissue and the material.

There are several attempts to solve this problem, for example by adding composite material or polymer(s) incorporating gradually degradable filler particles or leaching agents. Such materials have been described for example in the publications U.S. Pat. No. 5,324,775, EP 747 072 and WO 94/25521. The document U.S. Pat. No. 5,324,775 discloses biocompatible conjugates formed by covalently binding a biologically inactive polymer to hydrophilic polymers. The conjugate's hydrophilic part is polyethylene glycol or a derivative thereof having a weight average molecular weight from 100 to 20,000. The conjugates according to U.S. Pat. No. 5,324,755 may be used by directly injecting the components into the body whereafter the conjugate is formed in situ or by suspending the dried, particulate conjugate in a non-aqueous medium and further injecting said suspension into the body. In this latter embodiment, the medium is then removed by natural physiological conditions and the particles rehydrate and swell to their original shape. The composition comprising said conjugate may also contain biologically active proteins and/or particulate material suitable for bone repair purposes. In this latter case, the conjugate will form the matrix of the resulting composite.

In summary, the particles inside the polymer matrix can produce a porous structure by degradation. The continuous phase of the polymer matrix surrounds the resorbable filler particles, which particles form random voids in contact with body fluids. The porous structure is thereby formed by degradation of the filler particles. Remaining porous polymer matrix will give a framework for new tissue ingrowth and healing process. The continuous polymer matrix may be made of an absorbable (e.g. polyesters, polyanhydrides, polycarbonates) or a non-absorbable (e.g. acrylic polymer and its derivatives) biocompatible polymer or mixtures thereof.

Several publications also describe the use of a resorbable fillers in bone cements, such as for example WO 98/16268. During the formation of porosity inside the composites, the mechanical properties of the material dramatically decrease. Polymeric bioabsorbable particles, which are embedded inside the polymer matrix, degrade on contact with body fluid. Normally, the porous phase is able to form with difficulty, because in most cases the inert or slowly absorbable polymer matrix covers the outermost layer of well-embedded filler particles hindering or delaying the porosity formation. Thus formation is still restricted to the interface between the composite and the tissue, but not inside the composite material within a short time period after implantation.

As a summary, it can be said that although the degradation rate of different biocompatible polymers can be adjusted, the lack of porosity remains the major problem limiting their clinical applicability. Furthermore, another problem faced with the known porous materials having a polymeric matrix is the existence of at least a thin film of the polymer matrix between the pore and the surrounding tissue, a film that slows down the formation of the new tissue.

### OBJECT AND SUMMARY OF INVENTION

An object of the invention is to provide a material suitable for attaching, growing and/or repairing of living tissues and having an appropriate porosity throughout the density of the material. A further object of the invention is to provide a porous material wherein there is no polymer film between the pore and the surrounding tissue.

Another object of the invention is to provide a material wherein the porosity develops only after the material has been injected into the tissue to be treated. A yet further object of the invention is to provide a material having a continuous porous structure that facilitates the vascularisation of the bulk material, a feature that is essential for the growth of new tissue through the filled defect. A further object of the invention is to provide a material wherein the polymer matrix gives a framework for the healing process and it degrades totally only after the new tissue can withstand the external load. The invention further aims to provide a material suitable for attaching and/or growing living tissues.

The invention relates to a composite for attaching, growing and/or repairing of living tissues in mammals. The invention is characterized in that said composite comprises a non-expandable matrix polymer and a water-expandable porosity agent.

The invention further relates to the use of said composite in the manufacture of products for treatment of defects of tissue, for attaching tissues and/or growing tissues. The invention also relates to the use of the composite in implant, prosthesis, wound and/or tissue coating. The invention still relates to the use of the composite in the manufacture of reconstructive parts for tissues, tissue guiding membranes, bone augmentation materials, bone cements and/or scaffolds for tissue engineering.

### DETAILED DESCRIPTION OF THE INVENTION

The invention relates to a composite for attaching, growing and/or repairing of living tissues in mammals. The invention is characterized in that said composite comprises a non-expandable matrix polymer and a water-expandable porosity agent.

The matrix polymer of the present composite is said to be non-expandable. A person skilled in the art readily knows that virtually every polymer absorbs a small quantity of water and therefore, the term “non-expandable” in this connection is to be understood as being essentially non-expandable. In any case, the expansion coefficient of the matrix polymer is negligible compared to the expansion coefficient of the porosity agent.

The present invention thus provides a composite comprising a component that, once in contact with body fluids, expands (swells) and breakes the originally continuous phase of the matrix thus exposing the expanded component to the body fluids and reveals the bioactive part of the composite. The voids formed in the composite thereby come into contact with the surrounding tissue. There is in consequence no polymer film between the pore and the surrounding tissue, as in the prior art composites.

The composite according to the present invention thus provides a material wherein the outer-most surface is broken in order to form a continuous porous structure inside the material. The composite according to the present invention is thus capable of increasing the contact surface between the living tissue and the composite due to the phenomenon of expansion and thereafter porosity formation inside and on the surface of the composite. Thus, a continuous porous structure can rapidly be formed in the composite according to the present invention, either on the outermost layer of the matrix or throughout the composite. The porosity formation increases the bone ingrowth and in consequence, in the long-term strengthens the mechanical connection between the composite and the living tissue.

The composite according to the invention may thus be one wherein the porosity develops only after the material has been injected into the tissue defect to be repaired, or porosity can be formed prior to the introduction of the composite by a pre-treatment as will be discussed below.

However, the porosity formation will also at the same time decrease the flexural and compressive properties of the composite after being in contact with body fluid. The reduction will be greatest for the composites with the highest porosity, when the porosity agent has been well embedded into the matrix, in order to form a continuous phase inside it. In applications where such a decrease is undesirable, it can be compensated by cross-linking the matrix or by adding reinforcing fibers to the matrix.

The porosity agent may simultaneously dissolve and hydrolyze. Consequently, the molecular weight of the porosity agent may decrease, which in turn may speed up the pore formation.

The pores formed in the composite according to the invention can be either micro pores, e.g. having a diameter below 10 μm, or macro pores, e.g. having a diameter between 100 to 400 μm. By “diameter” in the case of non-spherical or irregularly shaped pores it is to be understood as meaning the longest axis that can be laid through the pore. Said pores may be spherical or tubular is shape.

The composite according to the invention is preferably biocompatible. The term “biocompatible” in this description relates to a material that is not deleterious to the recipient thereof.

According to an embodiment of the invention, the composite comprises from 1 to 99 wt-% of said water-expandable porosity agent and from 1 to 99 wt-% of said non-expandable matrix polymer, the total being 100%. It is obvious to a person skilled in the art that the amounts may be freely chosen and that they can be any amount between the above-identified limits. The amounts used are determined by the effect to be achieved with the composite and the location where it is used.

According to another embodiment of the invention, the non-expandable matrix polymer is bioresorbable and preferably selected from the group consisting of ε-caprolactone, polylactide and copolymers thereof. By the term “bioresorbabe”, it is meant materials that are biodebradable, biodissolvables, etc., i.e. materials that resorb in biological conditions (in contact with body fluids or living tissues).

A composite according to this embodiment is thus a material wherein the polymer matrix gives a framework for the healing process and resorbs totally only after the new tissue can withstand the external load.

According to yet another embodiment of the present invention, said non-expandable matrix polymer is on the contrary non-resorbable and preferably selected from the group consisting of polymethylmethacrylate, ethyleneglycoldimethacrylate, urethanedimethacrylate, butenedioldimethacryle, hydroxyethylenemethacrylate, bis-hydroxymethacryloxyphenylpropane, a hyperbranched methacrylate, methacrylate functionalized dendrimer and copolymers thereof. By dendrimer it is understood large spherical hyperbranched polymers.

According to a yet further embodiment of the invention, said water-expandable porosity agent is selected from the group consisting of collagen, derivatives of collagen, poly(ethylene glycol), poly(vinyl alcohol), polysaccharides, polyesters, celluloses, derivatives of cellulose, chiral polymers of hydroxyproline and mixtures thereof.

According to a preferred embodiment of the invention, said water-expandable porosity agent is a hydrolytic chiral polymer of hydroxyproline having a weight average molecular weight from 500 to 50000 g/mol. The hydroxyproline is preferably trans-4-hydroxy-L-proline. A more preferable weight average molecular weight is in the range from 5000 to 15000 g/mol.

According to a further preferred embodiment of the invention, said chiral polymer of hydroxyproline is a polyamide or polyester of trans-4-hydroxy-L-proline.

The above-mentioned chiral polymer of hydroxyproline, when used as the porosity agent, starts to resorb via hydrolysis immediately (in minutes) after becoming exposed to an aqueous environment. Resorption is completed within a few days, leaving a porous network within the bulk material releasing the admixed active agents directly into the tissue environment. By few days, it is meant 2 to 5 days, at most 7 days.

The resorption of the water-expandable porosity agent can be rapid or slow. The rate of resorption can be varied by the choice of the porosity agent and/or its molecular weight. The porosity agent may also be a blend of a water-expandable material and some other soluble material such as a sol-gel derived ceramic.

The preferred chiral polymer consisting of hydroxyproline used as the water-expandable porosity agent in the present invention was selected on the basis that:

(a) hydroxyproline is one of the amino acids in collagen molecule and it exists naturally in all mammalian tissues,

(b) synthetic chiral polymer of hydroxyproline degrades via hydrolysis,

(c) the hydrolysis begins immediately in aqueous environment and the chiral polymer is completely degraded within few days,

(d) the degradation products are non-toxic to mammals and

(e) bioactive components may be mixed with the chiral polymer.

As an example, a composite comprising a chiral polymer of hydroxyproline and ε-caprolactone-D,L-lactide may be prepared by grounding the chiral polymer of hydroxyproline in granules having a diameter of less than 500 μm after which the granules are incorporated within the ε-caprolactone-D,L-lactide, by melting the copolymer in a glass vial (warming it up to a temperature of 50° C.) and mixing the granules into the liquid copolymer.

According to another embodiment of the invention, the composite further comprises a bioactive agent as filler selected from the group consisting of drugs, mineralising agents, antimicrobial agents, bioactive glass, silica-gel, sol-gel derived ceramics, ormosiles (organic modified silica gels), hydroxylapatites, titanium-gel, growth factors, fluoride, heparin, anti-inflammatory agents, vitamins, tooth whitening agents, corticosteroids, living cells, preservatives, colouring agents, flow enhancing agents, bonding enhancing agents, suspension enhancing agents, mechanical properties enhancing agents and any combinations thereof. The amount of the bioactive agent may be freely selected from any amount between 1 to 99% of the total composition. The bioactive agent may be in any suitable form, for example in the form of particles, whiskers, granules, nets, microspheres and/or fibers. The bioactive agent may thus also be a reinforcing filler as mentioned above. The ratio of the porosity agent and said bioactive agent is such that the material remains homogenous during the application procedure. The term “homogenous” used herein is intended to include all compositions not bearing a risk of segregation of one or more of the components of the mixture when allowed to stand for long periods of time.

According to yet a further embodiment of the invention, said bioactive agent is located within the non-expandable matrix polymer, the water-expandable porosity agent, both of them and/or between layers of non-expandable matrix polymer and water-expandable porosity agent.

In general, the composite according to the present invention may be produced to a device consisting of a homogenous or unhomogeneous mixture of the components or of layers of components. When the device consists of several layers, the different layers may consist of different components having different resorption rates. A similar effect may also be obtained with an unhomogeneous distribution of the components in the composite. The composite according to the invention may also be in the form of an injectable material, such as a solution, a suspension, a thermoplastic material or a material consisting of granules. The material may thus be for example a thermoplastic material that has been warmed so that it has bocome liquid or it may be still in the form of monomers which polymerize once in contact with the tissue.

A composite according to the invention may be prepared by grounding the porosity agent into granules with a diameter of e.g. less than 500 μm and incorporating the granules into the matrix polymer by for example melting the polymer in a glass vial by warming it up to over its melting temperature and mixing the granules into the liquid polymer. One alternative method of preparation is to first form a porous structure of the porosity agent and a bioactive agent and then to incorporate the matrix polymer on that structure.

A device for repairing soft and hard tissue defects can then be manufactured by preparing a block of the above-mentioned composite and by tailoring it to the conditions (e.g. anatomical or geometrical) where it will be used. The device may further be pre-treated in an aqueous environment (e.g. simulated body fluid, cell/tissue culture medium) in order to create porosity before use. A pre-treated device can be implanted in hard or soft tissues, used as a drug delivery device, as a matrix for cell/tissue cultures or as a storage vial for cells/tissue components.

The composite according to the invention should also not be too flexible, in order to get the full benefit from the expansion of the porosity agent. Preferably, the Young's modulus of the non-expandable matrix polymer is from 1000 to 30000 MPa, more preferably from 1800 to 30000 MPa, at physiological temperature. The Young's modulus of the present composite may be modified by adding generally known fillers, such as the ones described above in connection with the bioactive agent.

The water-expandable porosity agent according to the present invention may also be used together with a bioactive agent as described above and in the absence of a polymeric matrix. Such a composition is especially suitable for coating of different materials such as titanium. The characteristics of said porosity agent and said bioactive agent in such a composition are identical to those listed above in connection with the inventive composite.

The invention also relates to the use of the composite according to the invention in the manufacture of products for treatment of defects of soft and hard tissue. The composite can also be used in reconstruction or augmentation of soft and hard tissue structures in a patient in need thereof by injecting the material into tissue defects directly.

According to an embodiment of the invention, the tissue to be treated is selected from the group consisting of maxilla, mandible, tooth, root canal, ear, nose, skull, joints, bone, subcutaneous tissue, intradermal tissue and dermal tissue.

According to another embodiment of the invention, the product is a dental product for root canal filling of a tooth or a cavity of a tooth, for sementing of temporary crowns, for periodontal packing for periodontal defects, for fitting of dentures, for occlusal splints, for mineralising splints, and/or for whitening of teeth.

The invention still relates to the use of the composite according to the invention in implant, prosthesis, wound and/or tissue coating. According to an embodiment of the invention, the tissue to be coated is selected from the group consisting of skin, cartilage, connective tissue, muscle, teeth and bone. The composite according to the present invention may also be used for different wounds, for example burns.

The invention further relates to the use of the composite according to the invention in the manufacture of reconstructive parts for tissues, tissue guiding membranes, bone augmentation materials, bone cements and/or scaffolds for tissue engineering. According to an embodiment of the invention, said reconstructive part for tissues is selected from the group consisting of bone filling blocks, granules, joints, sheets, rods, tubes, stents, fixation elements and pins. The composite according to the invention may be used in the form of an injectable material such as a solution, a suspension, a thermoplastic material or a material consisting of granules. The composite according to the invention may further be used either for producing endosseus prosthesis or for coating them at least partly. Examples of such endosseus prosthesis are hip and knee prosthesis.

### BRIEF DESCRIPTION OF THE DRAWINGS

FIGS. 1a, 1b and 1c schematically present the phenomenon of expansion of a composite according to a first embodiment of the invention.

FIGS. 2a, 2b and 2c schematically present the phenomenon of expansion of a composite according to a second embodiment of the invention.

FIG. 3a schematically discloses a composite according to a third embodiment of the invention.

FIG. 3b schematically discloses a composite according to a fourth embodiment of the invention.

FIG. 4a is a scanning electron microscope (SEM) micrograph of a composite according to a fifth embodiment of the invention.

FIG. 4b is a SEM-micrograph of the composite according to the fifth embodiment of the invention after immersion in simulated body fluid (SBF).

FIG. 5 is a micro computer tomograph (μ-CT) of a composite according to a sixth embodiment of the invention.

FIG. 6 is a SEM-micrograph of a composite according to a seventh embodiment of the invention.

FIG. 7a is a SEM-micrograph of a composite according to an eight embodiment of the invention.

FIG. 7b is a SEM-micrograph of a composite according to a ninth embodiment of the invention.

FIG. 8a is a schematic illustration of one possible use of the composite according to the invention.

FIG. 8b is a schematic illustration of another possible use of the composite according to the invention.

FIG. 9 is a schematic illustration of yet another possible use of the composite according to the invention.

FIG. 10 is a SEM-micrograph of a porous structure of an endoprosthesis.

### DETAILED DESCRIPTION OF THE DRAWINGS

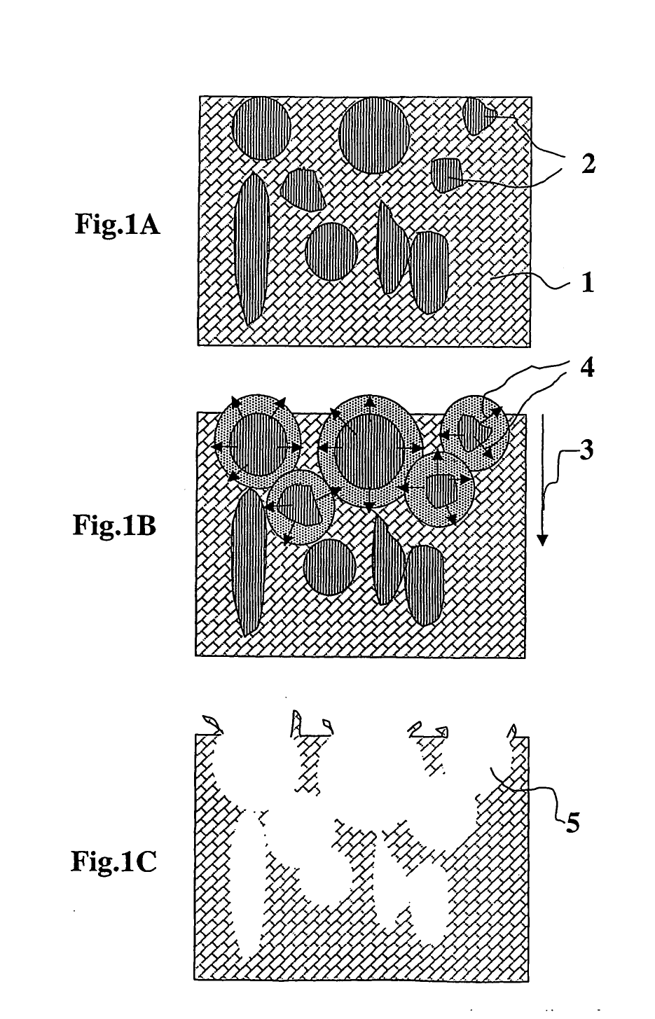

FIGS. 1a to 1c schematically disclose the phenomenon of expansion or swelling and porosity formation inside a composite according to a first embodiment of the invention, during the storage in an aqueous environment.

The composite as shown in FIG. 1a consists of two components, a non-expandable matrix polymer 1 and a water-expandable porosity agent 2. The porosity agent 2 is in the form of particles having various shapes and sizes. It may also be in the form of spheres or fibers, or any other form as will be readily understandable to a person skilled in the art.

FIG. 1b shows the phenomenon of expansion of the porosity agent 2 when the composite is stored in an aqueous environment. The arrow 3 shows the direction of the water sorption and the arrows 4 show the expansion of the porosity agent 2. The particles at the top of the Figure have already expanded and the particles at the bottom of the Figure will expand once they get into contact with water. The original shapes of the particles of the porosity agent are shown for clarity. The particles of the porosity agent expand to the extent that they get into contact with each other thus breaking the thin film of matrix polymer that subsists between particles in prior art composites, as described above. FIG. 1c presents the composite at the end of porosity formation, wherein random voids 5 have been formed as a consequence of the degradation of the expanded particles of the porosity agent. The porosity agent is thus biodegradable. The matrix polymer may be biodegradable or inert, and according to a preferred embodiment, if the matrix polymer is biodegradable, its degradation rate is smaller than the degradation rate of the porosity agent.

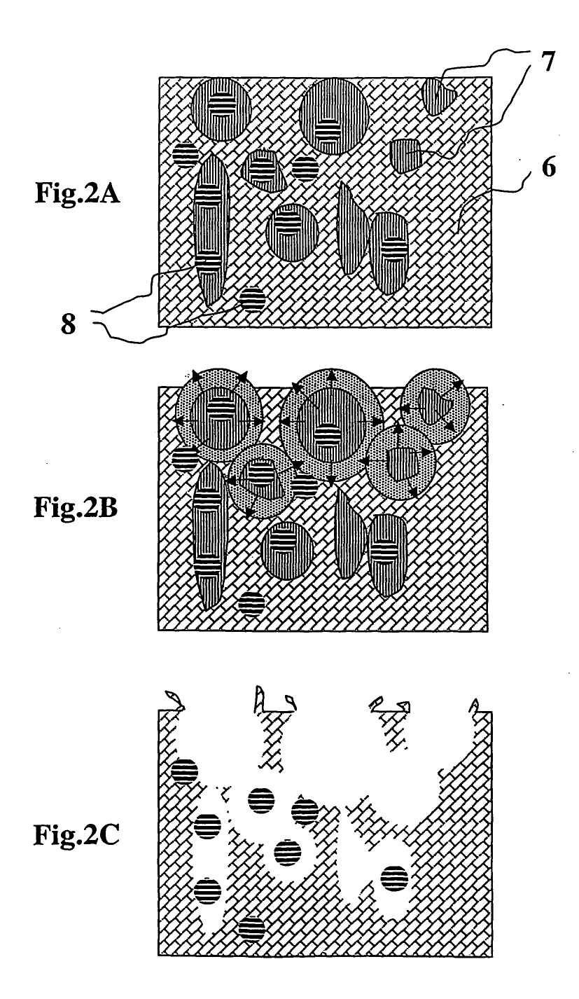

FIGS. 2a, 2b and 2c schematically present the phenomenon of expansion of a composite according to a second embodiment of the invention. The composite structure according to this invention comprises three components: a non-expandable matrix polymer 6, a water-expandable porosity agent 7 and a bioactive agent 8. The bioactive agent 8 may be mixed with one of the other components or with both of them, as in the present embodiment. The bioactive agent 8 in this embodiment is a bioactive glass in the form of granules. FIGS. 2b and 2c present the same phenomenon as FIGS. 1b and 1c, respectively. In FIG. 2c, it can be seen that the degradation rate of the bioactive agent 8 is preferably smaller than that of the porosity agent, thus leaving particles of the bioactive agent in the voids left by degraded porosity agent.

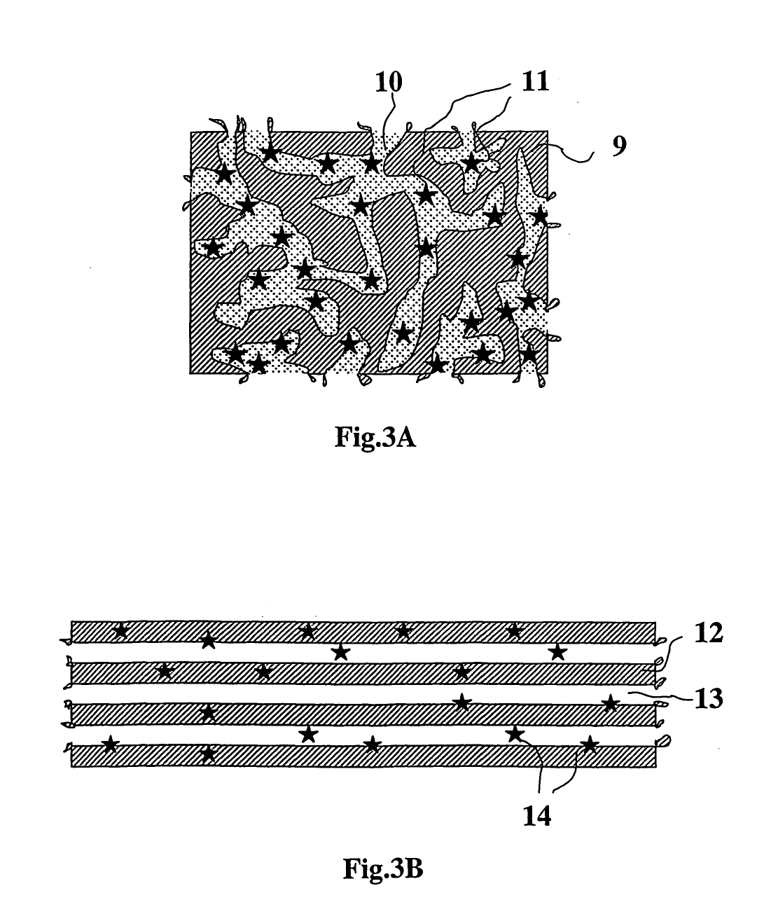

FIG. 3a schematically discloses a composite according to a third embodiment of the invention with two components 9 and 10 as well as a bioactive agent 11 mixed with the component 10. FIG. 3b schematically discloses a composite according to a fourth embodiment of the invention in which two components 12 and 13 form layers and a bioactive agent 14 is mixed with both components 12 and 13. This kind of construction of the component may be used for example in the form of a tube or a laminated sheet. The layers of such a construction may have different orientations.

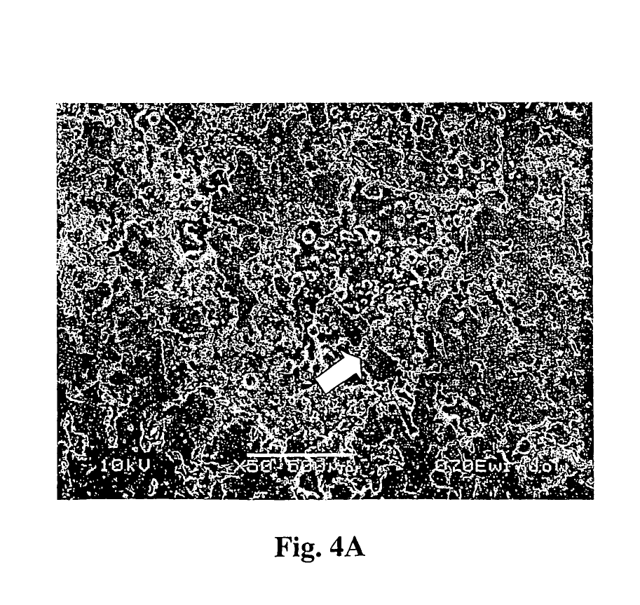

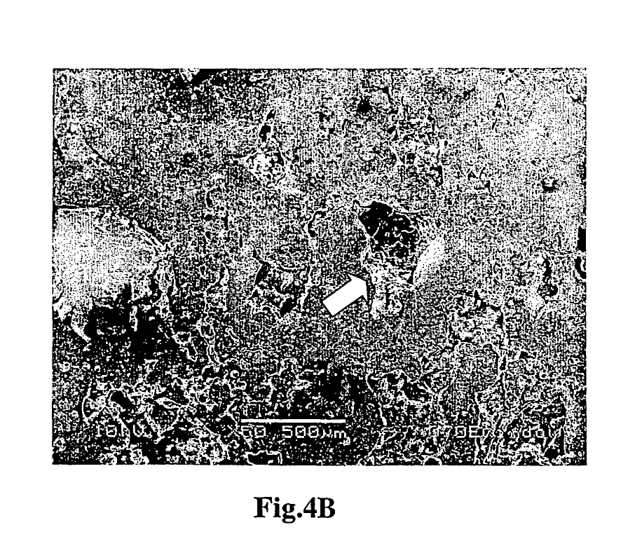

FIG. 4a is a SEM-micrograph showing the surface of a dry bone cement blend containing polyamide of trans-4-hydroxy-L-proline porosity agent (20 wt-%). The arrow shows separate phases of polyamide of trans-4-hydroxy-L-proline in the structure.

FIG. 4b is a SEM-micrograph showing the same bone cement blend as in FIG. 4a after storage in SBF. The Figure illustrates the porosity formation as a result of water sorption into the porosity agent. The arrow shows the dissolved phases of the polyamide of trans-4-hydroxy-L-proline.

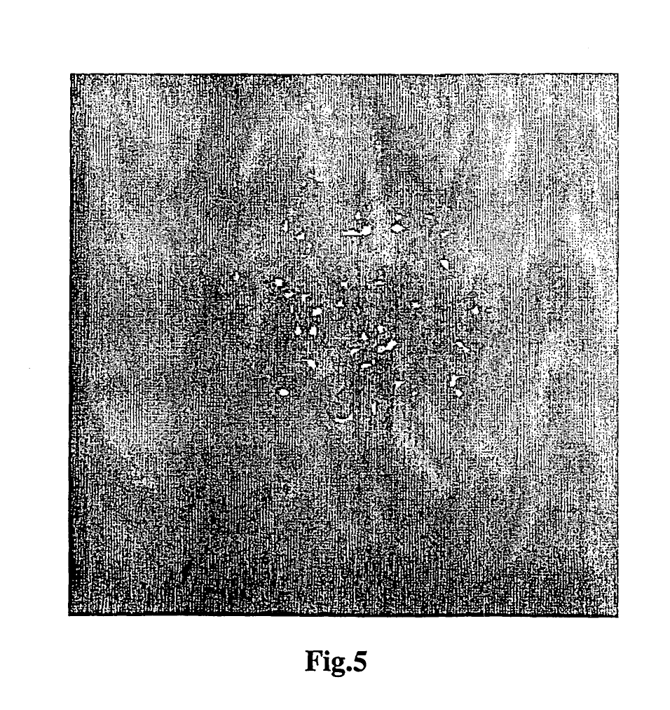

FIG. 5 is a micro computer tomograph (μ-CT) of a composite according to the sixth embodiment of the invention wherein the porosity agent is a chiral polymer and it has dissolved leaving a porous ε-caprolactone-D,L-lactide matrix.

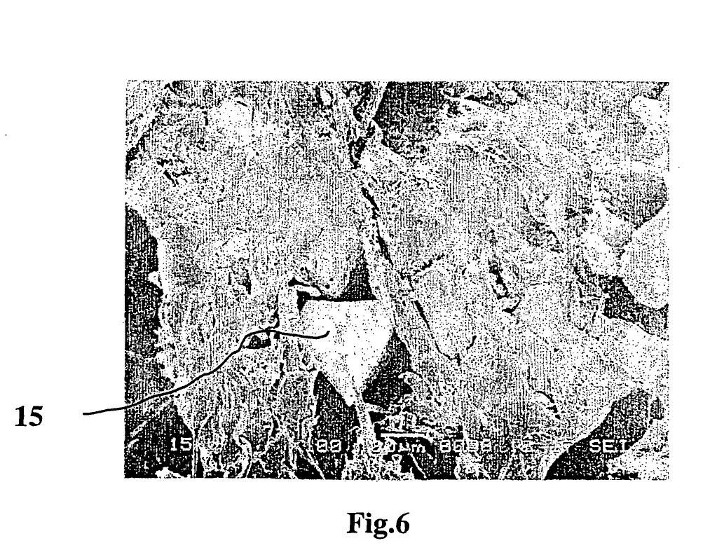

FIG. 6 discloses a SEM-micrograph of a composite according to the seventh embodiment of the invention. The composite according to this embodiment comprises a chiral polymer as porosity agent, a copolymer of ε-caprolactone-D,L-lactide as matrix and bioactive glass granules as bioactive component. The micrograph shows the composite wherein the bioactive component 15 has been exposed within the copolymer of ε-caprolactone-D,L-lactide after the chiral polymer has dissolved.

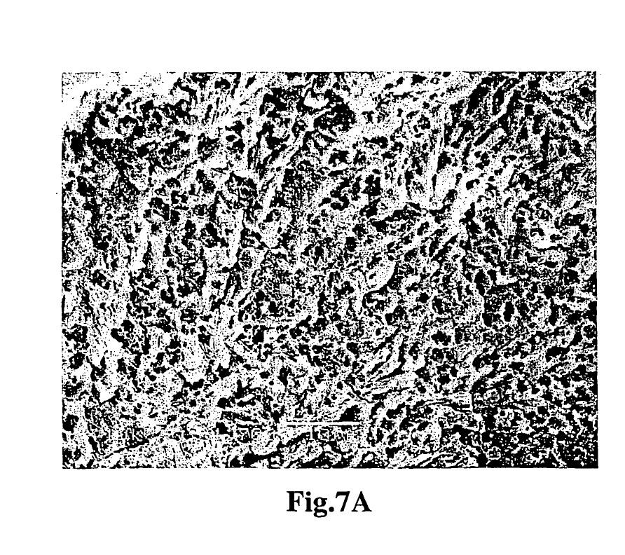

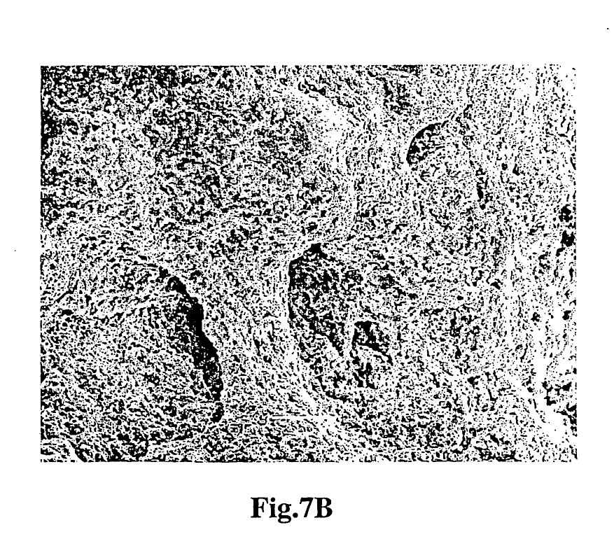

FIG. 7a discloses a SEM-micrograph of a porous structure formed by dissolving a porous polyester of trans-4-hydroxy-L-proline and FIG. 7b discloses a SEM-micrograph of a porous structure formed by dissolving a porous polyamide of trans-4-hydroxy-L-proline.

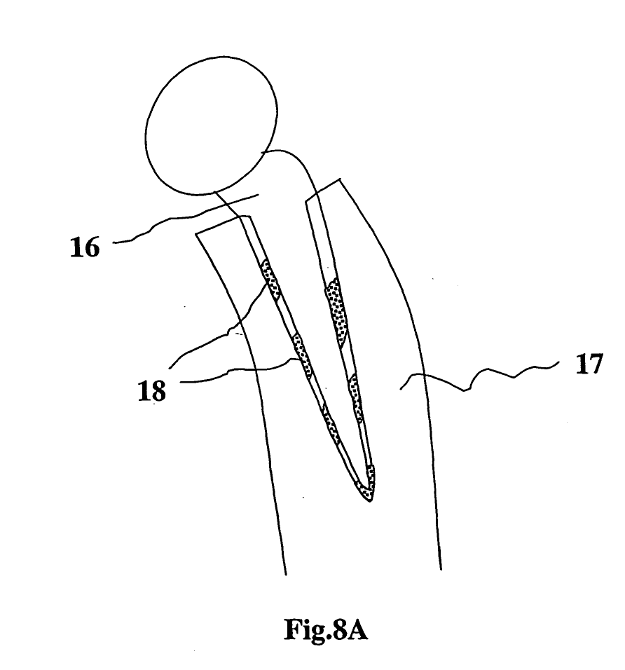

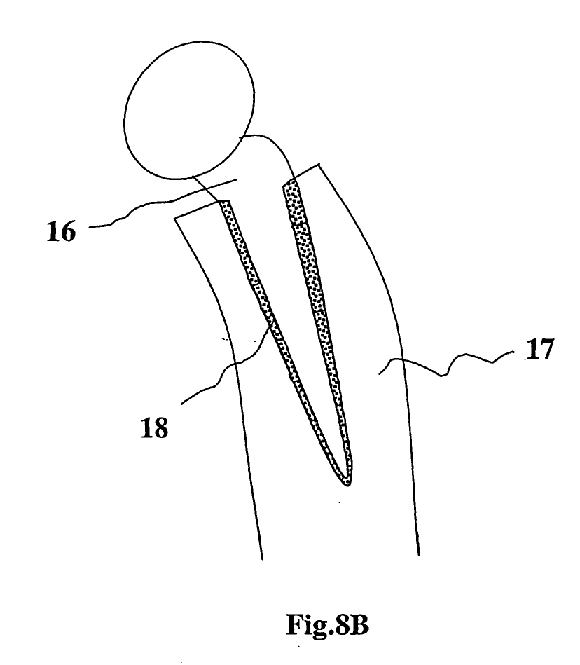

FIG. 5a is a schematic illustration of one possible use of the composite according to the invention. In this Figure, a hip-joint endoprothesis 16 in place in the femoral bone 17 is shown. The composite according to the invention is used as the bone cement 18 in the medullary canal. In this embodiment, said composite is used only in part of the medullary canal as bone cement, the rest of the canal being filled with some other material available to the skilled person. FIG. 8b is a schematic illustration of another possible use of the composite according to the invention, wherein said composite occupies the whole medullary canal.

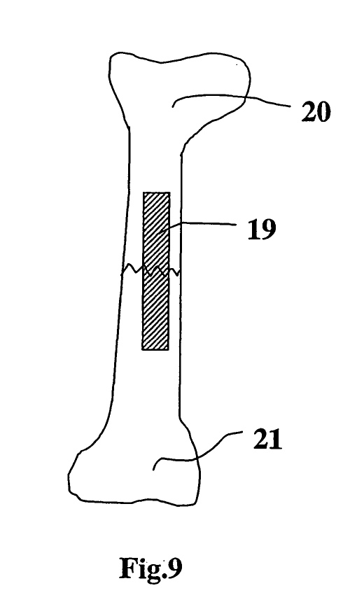

FIG. 9 is a schematic illustration of yet another possible use of the composite according to the invention. In this Figure, a sheet 19 made of the inventive composite is used to attach the parts 20 and 21 of a broken bone together.

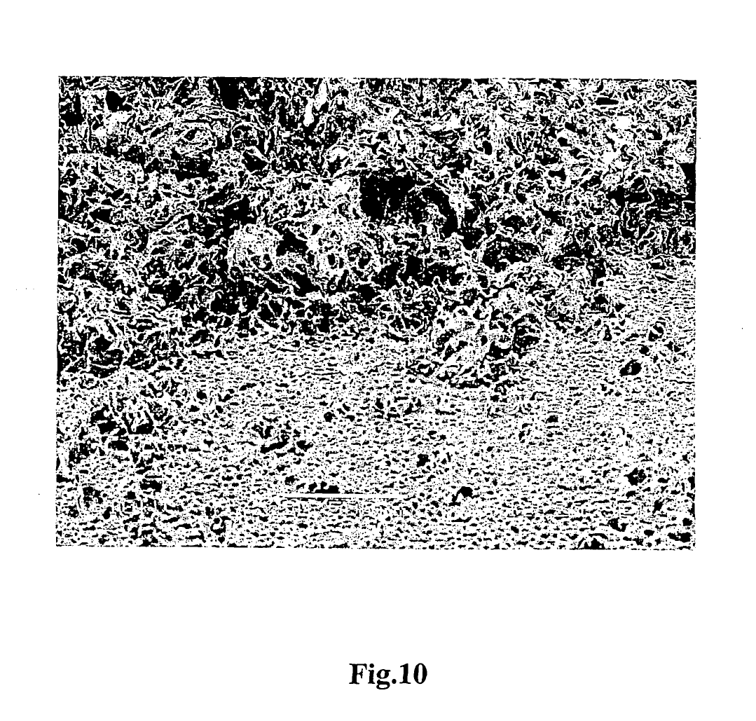

FIG. 10 is a SEM-micrograph of a porous surface of an endoprosthesis formed by the swelling/dissolving phenomenon as described in this application.

The following examples are given as illustrations of the present invention and are not to be construed as limitations thereof. Examples 1 and 2 concern the process for preparing water-expandable chiral polymers of hydroxyproline. Example 3 discloses the preparation of acrylic bone cement composite modified with a polyamide of trans-4-hydroxy-L-proline. Example 4 discloses the preparation and use of a composite according to one embodiment of the invention. Examples 5a and 5b demonstrate how the resorption, i.e. dissolving and degradation of the water-expandable polymer forms continuous pores in the composite. Examples 6 and 7 disclose composites with different bioactive agents. Example 8 describes a composite comprising bioactive glass and the use thereof. Example 9 describes a composition useful for coating implant materials. Example 10 discloses the preparation of acrylic bone cement composite modified with the polyamide of trans-4-hydroxy-L-proline AP(HP) and reinforced with glass fibers. Example 11 presents the preparation of acrylic bone cement composite modified with the polyamide of trans-4-hydroxy-L-proline AP(BP) and the crosslinking agent of ethyleneglycol dimethacrylate (EGDMA) and Example 12 the application of endoprothesis forming porous surface using swelling and dissolving the oligomer of polyamide of trans-4-hydroxy-L-proline AP(BP).

Experimental Part

### EXAMPLE 1

Chiral Polyamide of Hydroxyproline

Trans-4-hydroxy-L-proline methylester hydrochloride salt was synthesised from trans-4-hydroxy-L-proline (100 mol-%) in methanol and acetyl chloride (120 mol-%). Dried methanol was pre-cooled and stored in an ice/salt bath at 0° C., after which acetylchloride was added into the methanol extremely slowly, during a 30 minute period. Trans-4-hydroxy-L-proline was mixed with the dried methanol, and it was then added into a HCl-methanol mixture. The mixture obtained was stirred at a refluxing temperature under argon. Trans-4-hydroxy-L-proline methylester hydrochloride salt was a white crystalline solid.

Trans-4-hydroxy-L-proline methylester (monomer) was prepared from trans-4-hydroxy-L-proline methylester hydrochloride salt obtained by using an excess of the anionic ion exchange resin Amberlite IRA-400 ® (by Fluka) (OH-form, 20-50 mesh) in methanol. The solvent was evaporated. The monomer trans-4-hydroxy-L-proline methylester was obtained as a slightly viscous liquid.

A reaction flask was charged with said monomer. The reaction system was equipped with a N₂(liquid)/acetone trap. Initially the monomer was agitated during the first 5 min by flushing the system with nitrogen. The monomer was heated at 100° C., and the catalyst, calcium acetate (0.5 wt-%) was added. The reaction was heated further at 120° C. in high vacuum. The increase in molecular weight was monitored by measuring the viscosity throughout the reaction period. At the end of the reaction, the product, polyamide of trans-4-hydroxy-L-proline, appeared to be glassy and very hydrophilic.

### EXAMPLE 2

Chiral Polyester of Hydroxyproline

Trans-4-hydroxy-L-proline methylester hydrochloride salt was synthesised from trans-4-hydroxy-L-proline (100 mol-%) in methanol and acetyl chloride (120 mol-%). Dried methanol was pre-cooled and stored in an ice/salt bath at 0° C., after which acetylchloride was added into the methanol extremely slowly, during a 30 minute period. Trans-4-hydroxy-L-proline was mixed with dried methanol and added into the HCl-methanol mixture. The mixture obtained was stirred at a refluxing temperature under argon. The reaction mixture from the preparation of the methyl ester of trans-4-hydroxy-L-proline HCl-salt was cooled to 30° C. NaOH-solution (2 M, 120 mol-%) was added to the mixture. After this benzyl-chloride (120 mol-%) was added, and the mixture obtained was allowed to reflux for 1 h. Finally, NaOH-solution (2 M, 120 mol-%) was added at ambient temperature (25° C). A purified monomer, trans-4-hydroxy-N-benzyl-L-proline methylester was obtained as a viscous liquid.

A reaction flask was charged with this purified monomer. The reaction system was equipped with a N₂(liquid)/acetone trap. The monomer was heated at 120° C. and the catalyst, titanium isopropoxide (1 mol-%), was added. Initially the monomer was agitated every 15 min during the first hour by flushing the system with nitrogen to enhance mixing and to remove moisture. The reaction was heated further at 160° C. in high vacuum. The increase in molecular weight was monitored by measuring the viscosity throughout the reaction period. A brown, glassy and very brittle solid polyester of trans-4-hydroxy-N-benzyl-L-proline was obtained.

An autoclave was charged with this obtained polyester of trans-4-hydroxy-N-benzyl-L-proline, trifluoroethanol and palladium on charcoal (10%). The mixture was stirred at ambient temperature (25° C.) under hydrogen pressure (95 bar). At the end of the reaction, the catalyst was removed by filtration and the solvent evaporated. The product, polyester of trans-4-hydroxy-L-proline ester, appeared to be slightly hydrophilic and elastic.

### EXAMPLE 3

Preparation of acrylic bone cement composite modified with the polyamide of trans-4-hydroxy-L-proline AP(HP)

A commercial polymethylmethacrylate (PMMA) and polymethylmethacrylate- polymethylacrylate (PMMA-PMA) copolymer based bone cement (Palacos® R by Schering-Plough Labo n.v., Heist-op-den-Berg, Belgium) was used. Each dose of surgical bone cement consisted of 40 g of a PMMA-PMA copolymer and an ampoule with 18 g of methylmethacrylate (MMA) monomer. The mixture of PMMA-PMA/PMMA based bone cement with 20 wt-% of an experimental polyamide of trans-4-hydroxy-L-proline was used for the preparation of the test sample. The polymer powder (PMMA-PMA copolymer) was first mixed with the polyamide of trans-4-hydroxy-L-proline and the powder mixture was then mixed with the monomer solution (MMA) at room temperature. The blending of PMMA-PMA copolymer and polyamide of trans-4-hydroxy-L-proline powder together with MMA was accomplished by hand mixing for about 0.5 min. The bone cement resin mixture was polymerized by benzoylperoxide initiated and N,N-dimethyl-p-toluidine catalyzed autopolymerisation in air at room temperature for 15 min. The test sample was immersed in simulated body fluid (SBF) for one week at (37±1)° C.

### EXAMPLE 4

Preparation and Use of a Composite

A chiral polymer of hydroxyproline, having a molecular weight of about 10 000 g/mol, was melt and bioactive glass (S53P4, produced by Abmin Technologies, Turku, Finland) granules (particle size 91-310 μm) were mixed to form a 50:50 suspension of uniform consistency. The suspension obtained was then cooled down to room temperature and ground to granules with a mean diameter of <500 μm. These granules were then mixed with a thermoplastic ε-caprolactone-D,L-lactide copolymer in a 50:50 ratio. The composite was split in small pieces and packed into 5 ml syringes of which the narrowed tip had been cut of. The syringes were sterilized using gamma radiation, dose 25 kGy min. The syringe was heated up to 50° C. and the sterile composite was injected into a bone defect of a mammal. In the body of the mammal the dissolving of the chiral polymer creates canals in the copolymer matrix and exposes the admixed bioactive glass particles to the surrounding environment.

### EXAMPLE 5a

Porous Polyester of Trans-hydroxy-L-proline (HP)

Porous HP polymers can be produced by using a HP solvent and a HP non-solvent for coagulating HP. Used solvents are miscible with each other.

2,00 g of polyester of trans-hydroxy-L-proline (HP) was dissolved in 10 ml of isopropanol (by Sigma-Aldrich). The solution was poured into diethylether (by Sigma-Aldrich) causing HP to coagulate. After HP had coagulated in a porous form, it was impregnated with photocurable Sinfony Activator (by Espe, Dental-Medizin Gmbh &co. KG, Seefeld, Germany) and the mixture obtained was photocured. HP was then again dissolved thus forming a porous structure in the matrix polymer. The porous structure formed by dissolving the polyester of trans-4-hydroxy-L-proline is shown in the microradiograph of FIG. 4a.

### EXAMPLE 5b

Porous polyamide of trans-hydroxy-L-proline AP(HP)

Porous AP(HP) polymers can be produced by using a AP(HP) solvent and a AP(HP) non-solvent for coagulating AP(BP). Used solvents are miscible with each other.

0,5 g of polyamide of trans-hydroxy-L-proline AP(BP) was dissolved in 3 ml of isopropanol (by Sigma-Aldrich). The solution was poured into the mixture of tetrahydrofuran (by Sigma-Aldrich) and PMMA (25%) causing AP(HP) to coagulate in a porous form. The porous structure formed by dissolving the polyamide of trans-4-hydroxy-L-proline is shown in the microradiograph of FIG. 4b.

### EXAMPLE 6

Composite Doped with Ca and PO₄

An injectable composite was prepared as in Example 4 except that the bioactive glass was replaced with bioactive sol-gel derived ceramic filler doped with Ca and PO₄. The sol-gel derived ceramic was prepared according to the methods of the art such as taught in WO 97/45367 (Kangasniemi et al.). A similar composite as in example 4 was obtained.

### EXAMPLE 7

Composite Doped with Growth Factors

An injectable composite is prepared as in Example 4 except that the bioactive glass was replaced with bioactive sol-gel derived ceramic filler doped with growth factors. The sol-gel derived ceramic was prepared according to the methods of the art such as taught in WO 97/45367 (Kangasniemi et al.). A similar composite as in example 4 was obtained.

### EXAMPLE 8

A composite was prepared as in Example 4 after which the composite was compressed into moulds to form a membrane like device (thickness<0.8 mm). Another composite was made of ε-caprolactone-D,L-lactide and bioactive glass particles (S53P4 as in Example 4 above) as disclosed in WO 99/02201 (Aho et al.) and compressed to thin membranes (thickness<0.8 mm). A sandwich-like multilayer membrane was made by fusing three composite membranes together, leaving a chiral hydroxyproline membrane between the two ε-caprolactone-D,L-lactide/bioactive glass membranes. A bone defect in a rabbit scull is covered with the multilayer-membrane. The rapid dissolution of the hydroxyproline membrane left an empty space between the two membranes of bioactive glass containing ε-caprolactone-D,L-lactide. An apatite layer was formed in situ on the composite membrane surface that attracted osteoblast-like cells to migrate, attach, and mature on the surface of the newly formed apatite. The empty space between the membranes was gradually filled with new bone tissue.

### EXAMPLE 9

A composition comprising a chiral polymer and bioactive glass was prepared as described in Example 4 except that no matrix polymer was used. The composition was used to coat implant biocompatible materials, such as titanium. A similar composite as in example 4 was obtained.

### EXAMPLE 10

Preparation of acrylic bone cement composite modified with the polyamide of trans-4-hydroxy-L-proline AP(HP) and reinforced with glass fibers

A commercial polymethylmethacrylate (PMMA) and polymethylmethacrylate-polymethylacrylate (PMMA-PMA) copolymer-based bone cement (Palacos® R) was used. Each dose of surgical bone cement consisted of 40 g of a PMMA-PMA copolymer and an ampoule with 18 g of methylmethacrylate (MMA) monomer. Five groups of test specimens were prepared using the Palacos® R cement, the oligomer of polyamide of trans-4-hydroxy-L-proline AP(HP) filler and E-glass fibers (Stick Tech Ltd., Turku, Finland). Varying quantities of the oligomer of polyamide of trans-4-hydroxy-L-proline AP(HP), namely 5, 10, 15, and 20 wt-%, were used, said oligomer replacing a weight fraction of the bone cement. In the first group, the plain polymer powder (PMMA-PMA copolymer) was mixed with the monomer, methylmethacrylate (MMA), at room temperature. In the other groups, the polymer powder (PMMA-PMA copolymer) was first mixed with the oligomer of polyamide of trans-4-hydroxy-L-proline AP(HP) and the powder mixture was then mixed with the MMA solution at room temperature. E-glass fibers were used in two forms, one in continuous unidirectional (length 50 mm) and another in chopped form (length 2 mm). After the mixing of bone cement/the oligomer of polyamide of trans-4-hydroxy-L-proline AP(HP) with fibers, the mixtures were packed into rhomboidal-shaped moulds to prepare the specimens for a three-point bending test. Each test specimen with linear fiber reinforcing consisted of ca. 6,35 wt-% of fibers and a test specimen with chopped fiber reinforcing consisted of ca. 6,63 wt-% of fibers. The test specimens for study of the mechanical properties by the three-point bending were grouped according to the amount of the oligomer of polyamide of trans-4-hydroxy-L-proline AP(HP) (0-20 wt-%) added and the form of the fibers used. Each of these test specimen groups was divided into three subgroups, which consisted of six test specimens. The test specimens in Subgroup 1 were tested dry at room temperature (23±1)° C. The test specimens in Subgroup 2 were immersed in distilled water (volume V═50 ml) or in SBF (in Subgroup 3, V═50 ml) for one week at (37±1)° C. and tested in distilled water at (37±1)° C. The flexural strength and modulus of the bone cement reinforced and modified with the oligomer of polyamide of trans-4-hydroxy-L-proline AP(BP) filler was considerably higher compared to the non-reinforced specimens (see Tables I, II and III).

TABLE I
The mechanical properties of acrylic bone cement
with different quantities of the oligomer of polyamide
of trans-4-hydroxy-L-proline AP(HP).

| | Flexural Strength | | | Flexural Modulus | | |
|---|---|---|---|---|---|---|
| | (MPa) | | | (GPa) | | |
| | Dry | H₂O | SBF | Dry | H₂O | SBF |
| 0 wt-%¹ | 66 | 55 | 55 | 2.5 | 2.1 | 2.0 |
| 5 wt-% | 48 | 37 | 39 | 2.7 | 1.9 | 1.7 |
| 10 wt-% | 50 | 35 | 34 | 2.5 | 1.6 | 1.5 |
| 15 wt-% | 35 | 24 | 27 | 2.0 | 1.3 | 1.3 |
| 20 wt-% | 37 | 20 | 24 | 2.6 | 1.1 | 1.2 |

¹is the amount of the oligomer of polyamide of trans-4-hydroxy-L-proline AP(HP) filler.

TABLE II
The mechanical properties of acrylic bone cement with different quantities
of the oligomer of polyamide of trans-4-hydroxy-L-proline AP(HP) filler,
reinforced with continuous unidirectional fibers.

| | Flexural Strength | | | Flexural Modulus | | |
|---|---|---|---|---|---|---|
| | (MPa) | | | (GPa) | | |
| | Dry | H₂O | SBF | Dry | H₂O | SBF |
| 0 wt-%¹ | 145.3 | 94.5 | 106.9 | 4.6 | 3.5 | 4.0 |
| 5 wt-% | 135.3 | 82.5 | 91.4 | 4.7 | 3.7 | 3.6 |
| 10 wt-% | 127.8 | 85.6 | 82.2 | 4.3 | 3.5 | 3.9 |
| 15 wt-% | 130.0 | 78.0 | 79.8 | 4.4 | 3.5 | 3.9 |
| 20 wt-% | 117.7 | 65.6 | 68.9 | 4.2 | 3.0 | 3.4 |

¹is the amount of the oligomer of polyamide of trans-4-hydroxy-L-proline AP(HP) filler.

TABLE III
The mechanical properties of acrylic bone cement with different quantities
of the oligomer of polyamide of trans-4-hydroxy-L-proline AP(HP) filler,
reinforced with chopped random directional fibers.

| | Flexural Strength | | | Flexural Modulus | | |
|---|---|---|---|---|---|---|
| | (MPa) | | | (GPa) | | |
| | Dry | H₂O | SBF | Dry | H₂O | SBF |
| 0 wt-%¹ | 113.4 | 93.6 | 93.7 | 4.1 | 3.5 | 3.6 |
| 5 wt-% | 98.5 | 78.5 | 74.0 | 4.2 | 3.3 | 3.3 |
| 10 wt-% | 96.7 | 69.4 | 71.4 | 4.0 | 3.0 | 3.0 |
| 15 wt-% | 90.7 | 60.2 | 59.4 | 4.3 | 2.8 | 2.8 |
| 20 wt-% | 82.5 | 47.6 | 46.0 | 4.0 | 2.5 | 2.3 |

¹is the amount of the oligomer of polyamide of trans-4-hydroxy-L-proline AP(HP) filler.

The acrylic bone cement composite modified with the polyamide of trans-4-hydroxy-L-proline AP(HP) and reinforced with glass fibers can be used for example for cementing of hipjoint endoprothesis. In contact of body fluids, the polyamide of trans-4-hydroxy-L-proline AP(HP) swells, resorbs and forms finally the porosity inside the composites and the non-resorbable acrylic bone cement provides a framework for new tissue ingrowth.

### EXAMPLE 11

Preparation of acrylic bone cement composite modified with the polyamide of trans-4-hydroxy-L-proline AP(HP) and the crosslinking agent of ethyleneglycol dimethacrylate (EGDMA)

A commercial polymethylmethacrylate (PMMA) and polymethylmethacrylate-polymethylacrylate (PMMA-PMA) copolymer-based bone cement (Palacos® R) was used. Each dose of surgical bone cement consisted of 40 g of a PMMA-PMA copolymer and an ampoule with 18 g of methylmethacrylate (MMA) monomer. Four groups of test specimens were prepared using the Palacos® R cement containing 20 wt-% of the oligomer of polyamide of trans-4-hydroxy-L-proline AP(HP) and varying quantities (5, 10, 20, and 30 wt-%) of crosslinking agent EGDMA (ethyleneglycol dimethacrylate by Fluka). The polymer powder (PMMA-PMA copolymer) was first mixed with the oligomer of polyamide of trans-4-hydroxy-L-proline AP(HP) and the monomer of MMA and crosslinking agent of EGDMA were mixed together. After this, the powder mixture was added into the solution of monomer (MMA) and crosslinking agent (EGDMA) at room temperature. After the mixing, the composites were packed into rhomboidal-shaped moulds to prepare specimens for the three-point bending test. The test specimens for study of the mechanical properties by the three-point bending were grouped according to the amount of crosslinking agent (5-30 wt-%) added. Each of these test specimen groups was divided into two subgroups, which consisted of six test specimens. The test specimens in Subgroup 1 were tested dry at room temperature (23±1)° C. The test specimens in Subgroup 2 were immersed in SBF (V=50 ml) for one week at (35±1)° C. and tested in distilled water at (37±1)° C. The flexural strength and modulus of the oligomer of polyamide of trans-4-hydroxy-L-proline AP(HP) filler and crosslinking agent (EGDMA) modified bone cement are shown in Table IV.

TABLE IV
The mechanical properties of bone cement containing 20 wt-% of
oligomer of the polyamide of trans-4-hydroxy-L-proline AP(HP) and
various quantities of EGDMA crosslinker tested dry and after
immersion in SBF-solution for seven days.

| | |
|---|---|
| Flexural Strength | Flexural Modulus |
| (MPa) | (GPa) |

| | Dry | SBF | Dry | SBF |
|---|---|---|---|---|
| 5 wt-%¹ | 43.7 | 29.8 | 2.9 | 1.8 |
| 10 wt-% | 39.1 | 27.8 | 3.6 | 1.6 |
| 20 wt-% | 30.1 | 28.4 | 3.6 | 1.8 |
| 30 wt-% | 28.1 | 27.5 | 4.2 | 2.2 |

¹is the amount of the crosslinking agent (EGDMA).

### EXAMPLE 12

Application of endoprothesis forming porous surface using swelling and dissolving the oligomer of polyamide of trans-4-hydroxy-L-proline AP(HP), when in contact with body fluids

Strong non-resorbable fiber-reinforced composite core was manufactured using unidirectional long E-glass fibers prepreg having bisGMA/PMMA matrix (EverStick, by StickTech Oy, Turku, Finland). The FRC-core was photopolymerised in light curing oven for 15 min. Soon after polymerisation, the FRC-core was coated with a thin layer of PMMA, which had the polyamide of trans-4-hydroxy-L-proline AP(HP) (5:1 weight fraction) included in its matrix.

Such a FRC-endoprosthesis having PMMA the polyamide of trans-4-hydroxy-L-proline AP(HP) layer forms a porous surface layer when in contact with body fluids. At same time when surface porosity is formed, the polymer swelling fixes the endoprothesis to the bone.

It will be appreciated that the composite of the present invention can be incorporated in the form of a variety of embodiments, only a few of which are disclosed herein. It will be apparent for the specialist in the field that other embodiments exist and do not depart from the spirit of the invention. Thus, the described embodiments are illustrative and should not be construed as restrictive.

1. A composite for attaching, growing and/or repairing of living tissue in mammals, characterised in that said composite comprises a non-expandable matrix polymer and a water-expandable porosity agent.

2. The composite according to claim 1, characterised in that it is biocompatible.

3. The composite according to claim 1 or 2, characterised in that it comprises from 1 to 99 wt-% of said water-expandable porosity agent.

4. The composite according to to any of the preceding claims, characterised in that it comprises from 1 to 99 wt-% of said non-expandable matrix polymer.

5. The composite according to any of the preceding claims, characterised in that said non-expandable matrix polymer is biodegradable.

6. The composite according to claim 5, characterised in that said biodegradable matrix polymer is selected from the group consisting of ε-caprolactone, polylactide and copolymers thereof.

7. The composite according to claims 1-3, characterised in that said non-expandable matrix polymer is non-resorbable.

8. The composite according to claim 7, characterised in that said inert matrix polymer is selected from the group consisting of polymethylmethacrylate, ethyleneglycoldimethacrylate, urethanedimethacrylate, butenedioldimethacryle, hydroxyethylenemethacrylate, bis-hydroxymethacryloxyphenylpropane, a hyperbranched methacrylate, methacrylate functionalized dendrimer and copolymers thereof.

9. The composite according to any of the preceding claims, characterised in that said water-expandable porosity agent is selected from the group consisting of collagen, derivatives of collagen, poly(ethylene glycol), poly(vinyl alcohol), polysaccharides, polyesters, celluloses, derivatives of cellulose, chiral polymers of hydroxyproline and mixtures thereof.

10. The composite according to claim 9, characterised in that said water-expandable porosity agent is a chiral polymer of hydroxyproline having a weight average molecular weight from 500 to 50000 g/mol.

11. The composite according to claim 10, characterised in that said chiral polymer of hydroxyproline has a weight average molecular weight from 5000 to 15000 g/mol.

12. The composite according to claim 10 or 11, characterised in that said chiral polymer of hydroxyproline is a polyamide or polyester of trans-4-hydroxy-L-proline.

13. The composite according to any of the preceding claims, characterised in that the Young's modulus of the non-expandable matrix polymer is from 1000 to 30000 MPa.

14. The composite according to claim 13, characterised in that the Young's modulus of the non-expandable matrix polymer is from 1800 to 30000 MPa.

15. The composite according to any of the preceding claims, characterised in that it further comprises a bioactive agent selected from the group consisting of bioactive glass, silica-gel, ormosiles, hydroxylapatites, titanium-gel, antimicrobial agents, fluoride, heparin, anti-inflammatory agents, growth factors, vitamins, tooth whitening agents, corticosteroids and living cells.

16. The composite according to claim 15, characterised in that said bioactive agent is located within the non-expandable matrix polymer, the water-expandable porosity agent and/or between layers of non-expandable matrix polymer and water-expandable porosity agent.

17. The composite according to claim 15 or 16, characterised in that said bioactive agent is in the form of particles, whiskers and/or fibers.

18. The composite according to any of the preceding claims, characterised in that it is in the form of an injectable material.

19. The composite according to claim 18, characterised in that the injectable material consists of a solution, a suspension, a thermoplastic material or a material consisting of granules.

20. Use of the composite according to any of the preceding claims in the manufacture of products for treatment of defects of tissue, for attaching tissues and/or for growing tissue.

21. Use according to claim 20, characterized in that the tissue to be treated is selected from the group consisting of maxilla, mandible, tooth, root canal, ear, nose, skull, joints, bone, subcutaneous tissue, intradermal tissue and dermal tissue.

22. Use according to claim 20, characterised in that said product is a dental product for root canal filling of a tooth or a cavity of a tooth, for sementing of temporary crowns, for periodontal packing for periodontal defects, for fitting of dentures, for occlusal splints, for mineralising splints, and/or for whitening of teeth.

23. Use of the composite according to any of claims 1 to 19 in implant, prosthesis, wound and/or tissue coating.

24. Use according to claim 23, characterized in that the tissue is selected from the group consisting of skin, cartilage, connective tissue, muscle, teeth and bone.

25. Use of the composite according to any of claims 1 to 19 in the manufacture of reconstructive parts for tissues, tissue guiding membranes, bone augmentation materials, bone cements and/or scaffolds for tissue engineering.

26. Use according to claim 25, characterised in that said reconstructive part for tissues is selected from the group consisting of bone filling blocks, granules, joints, sheets, rods, tubes, stents, fixation elements and pins.
